(19) United States
(12) Patent Application Publication
(10) Pub. No.: US 2016/0220584 A1
(43) Pub. Date: Aug. 4, 2016

(54) AQUEOUS NASAL SPRAY COMPOSITION OF CORTICOSTEROIDS

(71) Applicant: Gavis Pharmaceuticals, Somerset, NJ (US)

(72) Inventor: Bala Chandran Nayar, Somerset, NJ (US)

(73) Assignee: GAVIS PHARMACEUTICALS, Somerset, NJ (US)

(21) Appl. No.: 14/611,240

(22) Filed: Jan. 31, 2015

(51) Int. Cl. A61K 31/57; A61K 45/06; A61K 9/00; A61K 31/56; A61K 47/36; A61K 47/32

(52) U.S. Cl. CPC A61K 31/57; A61K 47/36; A61K 47/32; A61K 9/0043; A61K 31/56; A61K 45/06

(57) ABSTRACT

A novel and improved aqueous nasal spray composition of corticosteroids is provided. The composition is characterized by containing either chitosan or polycarbophil as the bioadhesive or mucoadhesive agent. The invention also provides a method of alleviating or treating corticosteroid responsive conditions or disease by topically administering said aqueous nasal spray composition of corticosteroid to the nasal cavity.

### BACKGROUND OF THE INVENTION

(a) Field of the Invention

The present invention is directed to a novel aqueous nasal spray composition of corticosteroids. More particularly, the invention relates to aqueous nasal spray compositions of corticosteroids comprising chitosan or polycarbophil as a bioadhesive or mucoadhesive agent. The invention is further directed to the use of said composition for alleviating or treating corticosteroid responsive conditions such as diseases of the upper and lower airway passages.

(b) Description of the Related Art

Topical administration of active substances is a widely used method for treating nasal and ophthalmic conditions. Active substances which come into consideration are, for example, vasoconstrictors, such as xylometazoline; antiallergic agents, such as cromoglycic acid; H1 receptor antagonists, e.g., dimethindene maleate and olopatadine; prostaglandins, e.g., latanoprost and travoprost; and non-steroidal anti-inflammatory agents, e.g., bromfenac. Another group of widely used active substances are corticosteroids, such as mometasone, triamcinolone, beclomethasone, fluticasone, etc.

The indications in which a certain nasally administered drug is to be applied are well known in the art and easily accessible through any drug's prescribing information. For example, vasoconstrictors are used as nasal decongestants for alleviating the typical symptoms of common cold, like running nose, obstructed nose etc., or in rhinitis or sinusitis. Antiallergic agents and corticosteroids are, e.g., used in antiallergic conditions, e.g., hay fever, or in anti-asthmatic and anti-inflammatory conditions.

Corticosteroids have been approved to reduce inflammation of the upper and lower airways. For instance, intranasal corticosteroids exert a range of effects that inhibit mucosal inflammation, including (1) reducing inflammatory cell infiltration, (2) decreasing the number of basophils, eosinophils, neutrophils and mast cells in the nasal passages and their secretions, (3) reducing release of inflammatory signals from cells, (4) decreasing mucus production, (5) vasoconstriction, and (6) reducing edema.

Many efforts have been put forth in designing a safe and efficacious corticosteroid composition. Several corticosteroids have been successfully formulated as aqueous suspensions. However, suspension compositions may not be completely desirable in some circumstances. Solution compositions may offer certain advantages under particular conditions. It has been thought that a corticosteroid solution composition would have an unacceptable safety profile because of increased systemic absorption which could suppress the hypothalamic-pituitary-adrenal (HPA) axis function of patients.

Indian Patent Application No. 844/MUM/2014 discloses a micro-emulsion composition comprising a combination of azelastine hydrochloride and mometasone furoate.

U.S. Pat. No. 8,679,545 discloses oral composition of corticosteroid useful for preventing or alleviating symptoms of the inflammation associated with inflammatory disease and conditions of the gastrointestinal (GI) tract. The composition comprises a corticosteroid and a viscosity enhancing agent in the form of a coating.

U.S. Pat. Application Pub. No. 20130217657 teaches an ophthalmic composition comprising non-steroidal anti-inflammatory agents, optionally a corticosteroid and a mucoadhesive polymer.

PCT Application Pub. No. WO 2004105731 teaches a nasal composition comprising a decongestant and corticosteroid along with other ingredients including a bioadhesive material.

PCT Application Pub. No. WO 9938492 discloses a nasal composition of a vasoconstrictor or anti-allergic agent H1 receptor antagonist that remains in nasal cavities for a long time. The composition contains water soluble C₁-C₈ alkyl-cellulose derivatives.

PCT Application Pub. No. WO 2010042701 teaches nasal compositions comprising mometasone furoate and rheology-modifying agents.

Zhaolu Zhu et al. (Asian Journal of Pharmaceutical Sciences, Volume 8, Issue 4, August 2013, Pages 218-227) teaches use of thiolated polycarbophil in nasal mucosal drug delivery.

Roy et al. (Designed Monomers and Polymers, Volume 12, 2009, Pages 483-495) teaches that chitosan, a cationic polyelectrolyte, exhibits excellent mucoadhesive properties in a neutral or alkaline medium.

A major difficulty with topically applied compositions is, however, their duration of action. These composition are, by their nature, applied to body surfaces which may be prone to abrasion, or washing and flushing with bodily or applied fluids, such as tears, sweat or mucous. A particularly difficult situation for the use of topical preparations is in body cavities, such as the nasal passageways. This is because such cavities are typically coated in a mucous membrane which is non-adherent and turned over rapidly. In addition thick, viscous preparations can be difficult to apply effectively and are difficult to manufacture due to high viscosity preventing sterile filtration.

Nasal administration of corticosteroids in liquid form, e.g., in the form of drops, a solution or a spray—as opposed to nasal administration in gel form—is desirable inter alia because of a much better distribution of the active substances within the small diameter nasal cavities and an easier handling and dosing, e.g., in paediatric or geriatric patients.

Upon administration of liquid nasal formulations the patients often suffer from side-effects like burning, dryness, stinging of the nasal mucosa or sneezing. One of the reasons for these side effects is that liquids—in contrast to gels—normally do not remain in the nasal cavities for a long period of time but are washed out fast.

Further, existing corticosteroid nasal compositions are typically either low viscosity and short-lived or longer lived at the price of high viscosity. Furthermore, existing corticosteroid nasal compositions are often capable of containing only a low level of active agent, due to the poor compatibility between the base composition and the active agent. This low level of active agent results in a composition which rapidly loses effectiveness as it begins to dissipate from the site of action. A type of new thixotropic nasal spray suspension formulation of corticosteroids has been developed. The formulations become less viscous when shaken and sprayed, then return to a more viscous state after application. This time-dependent, reversible loss of viscosity under shear (e.g., shaking or spraying) can be quantified in the rheological property of thixotropy. Such formulations are currently sold under the brand names Nasonex®, Vancenase® AQ, Nasacort® AQ, Rhinocort® Aqua and Flonase®.

It is still of considerable value to provide new and improved nasal spray formulations of corticosteroids which are comparatively more bioadherent to mucosal surfaces than known formulations, which can be formulated as a low viscosity preformulation, and which can become adherent upon contact with the desired surface. Furthermore it would be a significant advantage if the formulation is protective, non-irritating, and shows reasonable resistance to wear and exposure to an aqueous ambient environment.

### SUMMARY OF THE INVENTION

The present invention provides the following aspects, subject-matters and preferred embodiments, which respectively taken alone or in combination, further contribute to solving the object of the present invention.

As described in further detail below, advantageously the topical aqueous compositions of corticosteroids according to the invention comprise a selective bioadhesive or mucoadhesive agent chitosan and/or polycarbophil along with one or more additional topically acceptable carriers.

In a first aspect, the invention provides an aqueous nasal spray composition comprising:
(a) one or more corticosteroids;
(b) chitosan and/or polycarbophil; and
(c) one or more topically acceptable carriers.

In another aspect, the nasal spray composition is essentially devoid of any additional active agent, preferably, devoid of a non-steroidal anti-inflammatory drug, vasoconstrictor or decongestant.

In another aspect, the nasal spray composition is essentially devoid of any additional bioadhesive or mucoadhesive agent, such as a cellulose derivative, polyvinyl pyrrolidone, polyvinyl alcohol, polyacrylamide, dextran, gellan gum, poloxamer, carrageenan, galactomannans, polyethylene glycol, starch, carbopol, maltodextrin and xanthan gum.

In another aspect, the osmolality of the nasal spray composition of the invention is less than 350 mOsm/kg.

In another aspect, the viscosity of the nasal spray composition of the invention is in the range of 1 to 100 cps.

In another aspect, the invention provides a topical aqueous nasal spray composition comprising:
(a) one or more corticosteroids;
(b) chitosan and/or polycarbophil; and
(c) one or more topically acceptable carriers,
   wherein the composition comprises from about 0.5% to about 5% by weight of the chitosan and/or polycarbophil.

Topically acceptable excipients in the topical aqueous solution are selected from the group comprising of preservatives, buffering agents, surfactants, and osmolality agents.

In another aspect, the invention provides a topical aqueous nasal spray composition comprising:
(a) one or more corticosteroids;
(b) chitosan and/or polycarbophil;
(c) about 0.05-0.5% w/w of at least one preservative;
(d) about 0.1-2% w/w of at least one buffering agent;
(e) about 0.005-0.5% w/w of at least one surfactant;
(f) at least one osmolality adjusting agent; and
(g) water.

In another aspect, the invention provides a topical aqueous nasal spray composition comprising:
(a) one or more corticosteroids;
(b) chitosan and/or polycarbophil;
(c) at least one preservative selected from the group consisting of benzalkonium chloride and phenyl ethyl alcohol;
(d) at least one buffering agent selected from the group consisting of sodium citrate, citric acid and sodium phosphate;
(e) at least one surfactant selected from the group consisting of polysorbate 80;
(f) at least one osmolality adjusting agent selected from the group consisting of sodium chloride; and
(g) water.

In another aspect, the invention provides a method of alleviating or treating an upper or lower respiratory tract condition or disease. The method comprises topically administering to a patient's nasal cavity an aqueous nasal spray composition consisting essentially of one or more corticosteroids, chitosan and/or polycarbophil, and one or more topically acceptable carriers.

Still other aspects and advantages of the invention will be apparent from the following detailed description of the invention.

### DETAILED DESCRIPTION OF THE INVENTION

The invention provides for an aqueous nasal spray composition of corticosteroid comprising a bioadhesive or mucoadhesive agent selected from chitosan or polycarbophil. The inventors of the present invention have unexpectedly found that the aqueous nasal spray composition of corticosteroid was found to be retained at the application site over a longer time in the nasal cavity than the compositions containing other bioadhesive or mucoadhesive agents such as hydroxypropyl methylcellulose or polyvinyl pyrrolidone. In particular, use of chitosan or polycarbophil was found to significantly improve the therapeutic benefit of corticosteroids in the topical treatment. Moreover, the safety and efficacy profile of the composition according to the present invention was found to be at least similar or improved compared to the existing corticosteroid formulations.

The present invention further addresses the problems known for the existing formulations and provides aqueous nasal spray compositions of corticosteroid which not only are retained at the application site for a longer time and possess a low viscosity but also moisturize the nasal mucosa and keep it sufficiently moisturized for a prolonged period of time. As a result, aqueous nasal solutions having an excellent safety and efficacy profile and prolonged moisturizing properties are obtained. Low viscosity of the composition of the present invention provides an additional advantage for providing ease of administration as well as manufacturing.

An increase in the interaction of the composition with the surface of the nasal tract may be measured by measuring the retention time of the material along a length of an internal nasal surface, wherein the retention time is increased in the presence of the excipients as compared to their absence. As used herein, in certain embodiments, an internal nasal surface includes a nasal mucosa and/or a nasal epithelium, all of which terms are used interchangeably herein. In another embodiment, an increased interaction may be measured by the decrease in physiological manifestations or symptoms of the disease or ailment to be treated.

The terms “bioadhesive” and “mucoadhesive” as used herein are defined as a substance which adheres to the nasal mucosa, preferably to a greater extent than hydroxypropyl methylcellulose or polyvinyl pyrrolidone.

The term “topically acceptable carrier” is used herein to refer to a carrier considered by those skilled in the pharmaceutical, food or cosmetic arts to be non-toxic when used topically, particularly in the nasal cavity.

The term “corticosteroid” refers to a class of compounds useful in treatment of inflammatory conditions, including those resulting from infection. Corticosteroids can include compounds that are naturally occurring, synthetic, or semi-synthetic in origin, and are characterized by the presence of a steroid nucleus of four fused ring structures.

Suitable corticosteroids which can be used in the nasal spray composition of the invention includes mometasone furoate, fluticasone propionate or furoate, triamcinolone, clobetasol, beclomethasone, budesonide, hydrocortisone, hydroxyltriamcinolone, alpha-methyl dexamethasone, dexamethasone-phosphate, beclomethasone dipropionates, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, mometasone, ciclesonide, loteprednol, flunisolide, and pharmaceutically acceptable salts and mixtures thereof. Preferred corticosteroids are mometasone furoate, fluticasone propionate or furoate, triamcinolone and clobetasol.

The amount of corticosteroid in the composition ranges from about 0.001% to about 5% by weight of the composition.

Chitosan is a bioadhesive cationic biopolymer comprising glucosamine and N-acetyl glucosamine. It is prepared by the deacetylation of chitin. In accordance with the present invention, the degree of deacetylation, which represents the proportion of N-acetyl groups which have been removed through deacetylation, should preferably be in the range of from about 40 to about 97%, more preferably be in the range of from about 60 to about 96% and most preferably be in the range of from about 70 to about 95%. The chitosan should preferably have a molecular weight in the range of from about 10,000 to about 1,000,000 Da, more preferably in the range of from about 30,000 to about 800,000 Da and most preferably in the range of from about 50,000 to about 600,000 Da.

By the term “chitosan” we include all derivatives of chitin, or poly-N-acetyl-D-glucosamine, including all polyglucosamines and oligomers of glucosamine materials of different molecular weights, in which the greater proportion of the N-acetyl groups has been removed through hydrolysis (deacetylation) and pharmaceutically acceptable organic and inorganic salts of chitosan. Suitable salts include, but are not limited to, nitrate, phosphate, acetate, hydrochloride, lactate, citrate and glutamate. Preferred salts are chitosan glutamate and chitosan hydrochloride. The most preferred salt is chitosan glutamate.

Chitosan derivatives and their salts are suitable for use in this invention. Chitosan derivatives may be prepared by bonding moieties to the hydroxyl or amino groups of chitosan and may confer the polymer with changes in properties such as solubility characteristics, charge density and mucoadhesiveness. For example, suitable chitosan derivatives prepared by bonding moieties to the hydroxyl groups of chitosan include esters, ethers or other derivatives formed by bonding acyl and/or alkyl groups with the hydroxyl groups. Examples include O-alkyl ethers of chitosan and O-acyl esters of chitosan. Other examples of chitosan derivatives include carboxymethyl chitosan (e.g., Thanou et al, J. Pharm. Sci., 90, 38-46, 2001), trimethylchitosan (e.g., Thanou et al, Pharm. Res., 17-27-31, 2000), thiolated chitosans (e.g., Bernkop-Schnurch et al, Int. J. Pharm., 260, 229-237, 2003) and piperazine derivatives (e.g., Holappa et al, Macromol. Biosci., 6, 139-144, 2006).

Chitosan suitable for use in the present invention may be obtained from various sources, including Primex, Iceland; NovaMatrix, Norway; Cognis, Germany; and Meron Biopoloymers, India.

Polycarbophil is a high-molecular-weight acrylic acid polymer cross-linked with polyalkenyl ethers or divinylglycol. It is described in U.S. Pat. No. 5,225,196 as a bioadhesive polymer suitable for sustained release of medicaments. The bioadhesive effect of polycarbophil is produced by the carboxylic acid groups binding to the mucosal surfaces via hydrogen bonding interaction.

The amount of chitosan or polycarbophil in the composition ranges from about 0.5% to about 5% by weight of the composition.

The nasal spray composition of the invention is essentially devoid of any additional active agent, preferably devoid of a non-steroidal anti-inflammatory drug, vasoconstrictor or decongestant.

In one embodiment, the nasal spray composition may further comprise one or more antihistaminic agents. Suitable antihistaminic agents may be selected from the group comprising olopatadine, azelastine, brompheniramine maleate, chlorpheniramine maleate, doxylamine succinate, phenindamine tartate, pheniramine maleate, promethazine maleate, pyrilamine maleate, thonzylamine hydrochloride, astemizole, azatadine, acrivastine, cetirizine, clemastine, cyclizine, carebastine, cyproheptadine, carbinoxanine, descarboethoxyloratadine, desloratadine doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, picumast, pyrilamine, promethazine, terfenadine, tripelennamine, temelastine, trimeprazine, triprolidine, diphenhydramine, oxatomide, setastine, tazifyline, phenyltoloxamine, and pharmaceutically acceptable salts and mixtures thereof. The amount of antihistaminic agent in the composition may range from about 0.001% to about 5% by weight of the composition.

The aqueous nasal spray composition may additionally comprise a topically acceptable carrier, such as a diluent, preservative, buffering agent, osmolality adjusting agent and surfactant to facilitate delivery of the nasal spray composition. Where delivery of the nasal spray composition in a droplet or spray form is desired, the topically acceptable carrier can comprise an aqueous carrier such as, for example, saline. Aqueous carriers can contain about 0.1% to about 2.0% by weight of a salt, e.g., sodium chloride. The nasal composition can be isotonic, i.e., it has the same osmotic pressure as blood and lacrimal fluid. Suitable non-toxic topically acceptable carriers are known to those skilled in the art. In one embodiment, the nasal spray composition has an osmolality of less than 350 mOsm/kg, and preferably in the range of 200 to 350 mOsm/kg.

Suitable preservatives which can be used in the aqueous nasal spray composition may be selected from the group comprising benzalkonium chloride, phenyl ethyl alcohol, potassium sorbate, benzyl alcohol, sorbic acid, benzethonium chloride, parabens, thimerosal, chlorobutanol and combinations thereof, with benzalkonium chloride and phenyl ethyl alcohol being preferred. The amount of preservative in the composition may range from about 0.05% to about 0.5% by weight of the composition.

Suitable buffering agents which can be used in the aqueous nasal spray composition may be selected from the group comprising phosphate, acetate, citric acid, sodium citrate, sodium phosphate and citrate-phosphate, with citric acid, sodium citrate and sodium phosphate being preferred. The amount of buffering agent in the composition may range from about 0.1% to about 2% by weight of the composition.

Suitable surfactants which can be used in the aqueous nasal spray composition may be selected from the group comprising anionic, cationic, non-ionic, or zwitterionic surfactants, such as, by way of non-limiting example, polysorbate (e.g., polysorbate 20, polysorbate 60, polysorbate 40, polysorbate 80, polysorbate 81, polysorbate 85, polysorbate 120), bile acids or their salts (e.g., sodium taurocholates, sodium deoxytaurocholates, chenodeoxycholic acid, and ursodeoxycholic acid), nonoxynol or polyoxyethylene glycol fatty acid esters, pluronic or poloxamers such as Pluronic F68, Pluronic L44, Pluronic L101, combinations thereof, or the like, with polysorbate being preferred. The amount of surfactant in the composition may range from about 0.005% to about 0.5% by weight of the composition.

Suitable osmolality adjusting agents which can be used in the aqueous nasal spray composition may be selected from the group comprising sodium chloride, potassium chloride, sorbitol, glycerol and mannitol, with sodium chloride being preferred. In an embodiment, the aqueous nasal spray composition has an osmolality of less than about 350 mOsm/kg, preferably in the range of 200 to 350 mOsm/kg.

In an embodiment, the aqueous nasal spray composition of corticosteroid described herein is prepared by a process, which comprises the steps of:
(a) mixing surfactant in water;
(b) adding preservative, buffering agent and osmolality adjusting agent and mixing after addition of each ingredient to the mixture of step (a);
(c) mixing corticosteroid to the mixture of step (b); and
(d) adding chitosan or polycarbophil to the mixture of step (c) and mixing under vigorous agitation.

The invention further provides a method of treating a corticosteroid responsive condition or disease in a patient by topically administering the aqueous nasal spray composition as substantially described herein.

Suitable corticosteroid responsive conditions or diseases can be an upper or lower respiratory tract condition or disease, allergic or non-allergic rhinitis, sinusitis, infection or inflammation of the nasal mucous membrane.

In an embodiment, the method of alleviating or treating an upper or lower respiratory tract condition or disease comprising topically administering to a patient's nasal cavity an aqueous nasal spray solution composition consisting essentially of one or more corticosteroids, chitosan and/or polycarbophil, and one or more topically acceptable carriers.

For nasal administration of the nasal spray composition, various devices are available in the art for the generation of drops, droplets and sprays. For example, the nasal spray composition can be administrated into the nasal passages of a subject by means of a dropper (or pipet) that includes a glass, plastic or metal dispensing tube. Fine droplets and sprays can be provided by an intranasal pump dispenser or squeeze bottle as is well known in the art.

Other means for delivering the nasal spray composition, such as inhalation via a metered dose inhaler (MDI), may also be used according to the present invention. Several types of MDIs are regularly used for administration by inhalation. These types of devices can include breath-actuated MDIs, spacer/holding chambers in combination with the MDI, and nebulizers. The term MDI as used herein refers to an inhalation delivery system comprising, for example, a canister containing an active agent dissolved or suspended in a propellant optionally with one or more excipients, a metered dose valve, an actuator, and a mouthpiece. The canister is usually filled with a solution or suspension of an active agent, such as the nasal spray composition, and a propellant, such as one or more hydrofluoroalkanes. When the actuator is depressed a metered dose of the solution is aerosolized for inhalation. Particles comprising the active agent are propelled toward the mouthpiece where they may then be inhaled by a subject.

### Example 1

### Corticosteroid Aqueous Nasal Spray

TABLE 1

| Sr. | | Formula 1 | Formula 2 | Formula 3 | Formula 4 |
|---|---|---|---|---|---|
| No. | Ingredients | (% w/w) | (% w/w) | (% w/w) | (% w/w) |
| 1 | Corticosteroid | 0.1 | 0.08 | 0.05 | 0.01 |
| 2 | Preservative | 0.02 | 0.02 | 0.02 | 0.02 |
| 3 | Buffering agent | 1 | 0.75 | 0.5 | 0.3 |
| 4 | Surfactant | 0.03 | 0.02 | 0.01 | 0.01 |
| 5 | Chitosan/ | 2 | 1.75 | 1.5 | 1.25 |
| | Polycarbophil | | | | |
| 6 | Osmolality | Q.S. | Q.S. | Q.S. | Q.S. |
| | adjusting agent | | | | |

| | | |
|---|---|---|
| 7 | Purified Water | Q.S. to 100% |

### Process:

Sufficient quantities of purified water was taken in a stainless vessel equipped with a mixer and homogenizer. Surfactant (e.g., Polysorbate 80) was added and mixed in purified water. A preservative (e.g., benzalkonium chloride, phenoxyethanol), buffering agent (e.g., sodium dihydrogen phosphate), and osmolality agent (e.g., sodium chloride) were added and mixed in the vessel after addition of each ingredient. The corticosteroid was then added in the prepared mixture and mixed well. Finally, chitosan or polycarbophil was added to the mixture under vigorous agitation.

The resulting composition may be used for nasal administration by a nasal spray device to generate drops, droplets or sprays. For example, the composition can be filled into an intranasal pump dispenser or squeeze bottle.

1. An aqueous nasal spray composition comprising:
(a) one or more corticosteroid as the sole active ingredient;
(b) chitosan and/or polycarbophil; and
(c) one or more topically acceptable carriers.

2. The nasal spray composition of claim 1, wherein said composition is devoid of a non-steroidal anti-inflammatory drug and a decongestant.

3. The nasal spray composition of claim 1, wherein said corticosteroid is selected from the group consisting of mometasone, fluticasone, triamcinolone, clobetasol, beclomethasone, budesonide, flunisolide, halobetasol, flunidolone, dexamethasone, diflorasone, ameinonide, flurandrenolide, desonide or pharmaceutically acceptable salts thereof.

4. The nasal spray composition of claim 3, wherein said corticosteroid is selected from mometasone, fluticasone, and triamcinolone.

5. The nasal spray composition of claim 1, wherein said corticosteroid is present in the range from about 0.001% to about 5% by weight of the composition.

6. The nasal spray composition of claim 1, wherein said chitosan and/or polycarbophil is present in the range from about 0.5% to about 5% by weight of the composition.

7. The nasal spray composition of claim 1, wherein the osmolality of the composition is less than 350 mOsm/kg.

8. The nasal spray composition of claim 1, wherein the viscosity of the composition is in the range of 1 to 100 cps.

9. The nasal spray composition of claim 1, wherein said topically acceptable carriers are selected from the group comprised of preservative, buffering agent, surfactant, and osmolality agent.

10. The nasal spray composition of claim 1, wherein the composition is in the form of a suspension or solution.

11.-14. (canceled)

15. The aqueous nasal spray composition of claim 1, consisting essentially of:
(a) one or more corticosteroids as the active agent;
(b) chitosan and/or polycarbophil as the bioadhesive or mucoadhesive agent; and
(c) one or more topically acceptable carriers.

16. The aqueous nasal spray composition of claim 15, wherein the composition further consists essentially of at least one preservative, at least one buffering agent, at least one surfactant and at least one osmolality adjusting agent.

17. The aqueous nasal spray composition of claim 1, wherein the topically acceptable carrier is water.

18. An aqueous nasal spray composition consisting essentially of:
(a) one or more corticosteroids as the active agent;
(b) chitosan and/or polycarbophil as the bioadhesive or mucoadhesive agent;
(c) about 0.05-0.5% w/w of at least one preservative;
(d) about 0.1-2% w/w of at least one buffering agent;
(e) about 0.005-0.5% w/w of at least one surfactant;
(f) at least one osmolality adjusting agent; and
(g) water.

19. A method of treating corticosteroid responsive condition or disease in a patient comprises topically administering the nasal spray composition of claim 1.

20. A method of alleviating or treating upper or lower respiratory tract condition or disease comprising topically administering to a patient's nasal cavity the nasal spray composition of claim 1.

21. The aqueous nasal spray composition of claim 1, consisting essentially of:
(a) one or more corticosteroid as the sole active ingredient;
(b) chitosan and/or polycarbophil as the bioadhesive or mucoadhesive agent; and
(c) one or more topically acceptable carriers.
